(19) United States
(12) Patent Application Publication
(10) Pub. No.: US 2019/0172363 A1
(43) Pub. Date: Jun. 6, 2019

(54) Real-time assessment of an emotional state

(71) Applicant: NFACTORIAL ANALYTICAL SCIENCES PVT. LTD, Bangalore (IN)

(72) Inventor: Krishnan Arun, Bangalore (IN)

(21) Appl. No.: 15/998,494

(22) PCT Filed: Feb. 15, 2017
(86) PCT No.: PCT/IN2017/000040

§ 371 Date: Aug. 16, 2018

(30) Foreign Application Priority Data
Feb. 16, 2016 (IN) 201641005288

(51) Int. Cl. G09B 7/06; G06F 16/26; G16H 20/70

(52) U.S. Cl. CPC G09B 7/06; G06Q 10/105; G16H 20/70; G06F 16/26

(57) ABSTRACT

The invention provides a method and a system for assessment of an emotional state of users in a network. The method includes identifying an event in the network, presenting a status specific to the event to the plurality of users, receiving a response from each of the users corresponding to the presented status, analyzing the responses received to obtain a cumulative annotated emotional state and displaying the annotated emotional state for assessment of the status presented. A user interface is also provided.

### FIELD OF INVENTION

The invention generally relates to the field of human resource management and particularly to a method and a system for real-time assessment of an emotional state.

### BACKGROUND

Employee engagement is a property of relationship between the organization and its employees. Employee engagement plays a vital role in the overall development of an organization by significantly contributing towards organizational and individual performances. There are methods available in the art for determining employee engagement. One such method determines employee engagement by detecting an emotional state of a user. The modes of detecting emotional state include but are not limited to inputs from audio or video devices, inputs from sensors capable of sensing biometric data and analysis of digital data relevant to the user which include mail conversations and browse history.

Another method determines employee engagement by utilizing user emotional state in a business process. The method includes receiving indications of the emotional states of users interacting with at least one part of at least one business process; and identifying at least one problematic part of the at least one business process based on the received indication of the emotional states of the users. The method further includes the steps of generating statistical data based on the received emotional states and generating a notification regarding the at least one problematic business process part.

There is a need for a method that provides improved impact on performance outcomes in terms of profitability, productivity, turnover, safety incidents and quality.

### BRIEF DESCRIPTION OF DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows a flow chart depicting a method for real-time assessment of an emotional state of users in a network, according to an embodiment of the invention.

FIG. 2 shows a system for real-time assessment of an emotional state, according to an embodiment of the invention.

FIG. 3(a) illustrates a happiness index overview of the real-time emotional state assessment system, according to an embodiment of the invention.

FIG. 3(b) illustrates a contextual overview of the real-time emotional state assessment system, according to an embodiment of the invention.

FIG. 3(c) illustrates a comparison overview of the real-time emotional state assessment system, according to an embodiment of the invention.

FIG. 3(d) illustrates Intervention settings of the real-time emotional state assessment system, according to an embodiment of the invention.

FIG. 3(e) illustrates a graphical representation of text based feedback, according to an embodiment of the invention.

FIG. 3(f) illustrates a graphical representation of trending, according to an embodiment of the invention.

FIG. 3(g) illustrates a text network, according to an embodiment of the invention.

FIG. 3(h) illustrates a representative screenshot of an employee dashboard, according to an embodiment of the invention.

FIG. 3(i) illustrates strengths and weaknesses of employee happiness, according to an embodiment of the invention.

FIG. 3(j) illustrates strengths and weaknesses of employee engagement, according to an embodiment of the invention.

FIG. 3(k) illustrates a representative screenshot of a customer dashboard, according to an embodiment of the invention.

FIG. 3(l) illustrates a representative screenshot of Net Promoter Score Analysis, according to an embodiment of the invention.

### SUMMARY OF THE INVENTION

One aspect of the invention discloses a method for assessment of an emotional state of users in a network. The method includes identifying an event in the network, presenting a status specific to the event to the plurality of users, receiving a response from each of the users corresponding to the presented status, analyzing the responses received to obtain a cumulative annotated emotional state and displaying the annotated emotional state for assessment of the status presented.

Another aspect of the invention provides a system for real-time assessment of an emotional state. The system includes a first device configured for broadcasting a status, at least one second device capable of receiving the broadcasted status wherein the second device is configured for submitting a response with respect to the broadcasted status, an analyzer communicatively coupled to the first device and the second device wherein the analyzer is configured for analyzing the plurality of responses received and a display means coupled to the analyzer for displaying the analyzed responses.

Yet another aspect of the invention provides a user interface for real-time emotional state assessment. The user interface includes means for authenticating the user, displaying a cumulative annotated emotional state upon authentication of the user, presenting the user with a plurality of filters to analyse the emotional state and assessing the analysed emotional state real-time.

### DETAILED DESCRIPTION OF THE INVENTION

Various embodiments of the invention provide a method and a system for assessment of an emotional state of users in a network. The method includes identifying an event in the network 101, presenting a status specific to the event to the plurality of users 103, receiving a response from each of the users corresponding to the presented status 105, analyzing the responses received to obtain a cumulative annotated emotional state 107 and displaying the annotated emotional state for assessment of the status presented 109.

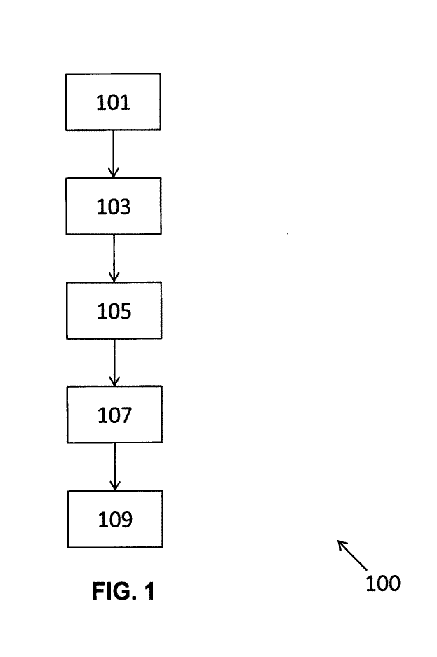

FIG. 1 shows a flow chart 100 depicting a method for real-time assessment of an emotional state of users in a network, according to an embodiment of the invention. The method includes identifying an event in the network. The event described herein includes but is not limited to a weather forecast, a sales promotion, a pay-hike, a fashion trend, a political event, a maternity leave, a lay-off, customer centricity, Manager Performance, Peer Group, Culture, Recreation, Diversity, Communication, Customer Orientation, Quality Emphasis, Training and Customer Satisfaction. The network described herein is a closed group network or an open network. Upon identification of the event, the plurality of users is presented with a status specific to the event. The users include but are not limited to employees, contract workers, customers, social media consumers, social employee networks and general public. The status is presented to the plurality of users iteratively over pre-determined intervals of time. The status described herein belongs to a multiplicity of classes wherein the classes include but are not limited to Employee, Performance and Customer centricity. The classes in turn are comprised of a multiplicity of contexts wherein the contexts include but are not limited to Fairness, Credibility, Trust, Growth and Development, Pay and Benefits, Work-Life Balance, Stress, Leadership, Manager Performance, Peer Group, Culture, Recreation, Diversity, Communication, Customer Orientation, Quality Emphasis, Training, Involvement and Customer Satisfaction.

The method further includes receiving a response from each of the users corresponding to the presented status. The response from the user is an emotional state that includes but is not limited to affection, anger, angst, anguish, annoyance, anxiety, apathy, arousal, awe, boredom, confidence, contempt, contentment, courage, curiosity, depression, desire, despair, disappointment, disgust, distrust, dread, ecstasy, embarrassment, envy, euphoria, excitement, fear, frustration, gratitude, grief, guilt, happiness, hatred, hope, horror, hostility, hurt, hysteria, indifference, interest, jealousy, joy, loathing, loneliness, love, lust, outrage, panic, passion, pity, pleasure, pride, rage, regret, remorse, sadness, satisfaction, schadenfreude, self-confidence, shame, shock, shyness, sorrow, suffering, surprise, trust, wonder, worry, zeal and zest.

The response is submitted as at least one of a picture, a text or an emoticon. The responses from the users are then analyzed to obtain a cumulative annotated emotional state. The analysis of the responses submitted is performed based on a plurality of parameters derived from the responses. The parameters described herein include but are not limited to nature of the user, time of response submission, date of response submission and location of user. The cumulative annotated emotional state described herein include but are not limited to net happiness score, happiness index, net emotion score, net emotion index, attrition score, productivity score, performance score, net employee promoter score and safety score. The cumulative annotated emotional state is presented as at least one of a graph, a pie-chart, a nested chart, a web-chart, radial chart, spider chart, bullet chart and using any relevant visual technique. The assessment is performed by at least one person concerned with the user. The person described herein includes but is not limited to a Manager, a CXO, a Team leader, a consumer marketing professional, a customer service associate, a customer experience leader, a human resource associate and a business partner.

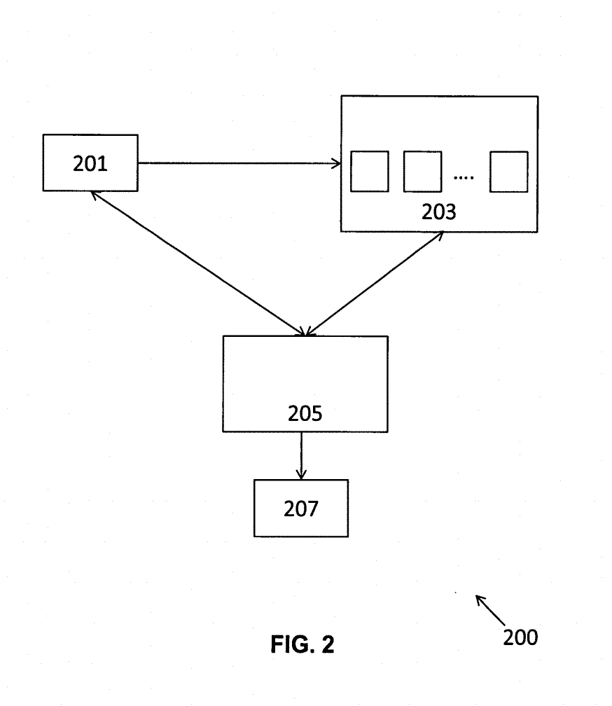

FIG. 2 shows a system 200 for real-time assessment of an emotional state, according to an embodiment of the invention. The system includes a first device 201 and at least one second device 203. The first device 201 is configured for broadcasting a status. In an embodiment of the invention, the broadcast of status by the first device is a push notification. The first device described herein includes but is not limited to a server, a desktop, a laptop or a handheld device. The second device 203 is configured for receiving the broadcasted status and submitting a response with respect to the broadcasted status. In an embodiment of the invention, the submitting of response by the second device is a push notification. The second device 203 described herein is a handheld device which includes but is not limited to a smartphone, a tablet, a fablet or a wearable device. An analyzer 205 is communicatively coupled to the first device 201 and the second device 203. The analyzer 205 is configured for analyzing the plurality of responses received. The analyzer 205 includes a database 209, an evaluator engine 211 communicatively coupled to the database 209 and a presentation module 213. The database 209 is configured for retrievably storing the plurality of responses. The database 209 described herein includes but is not limited to a physical storage or a virtual storage. The evaluator engine 211 is configured for real time analysis of the responses received. The presentation module 213 is configured to present the analyzed responses in at least one format which includes but is not limited to a table, a static graph, an index, an interactive graph, a pie-chart. A display means 207 is coupled to the analyzer 205 for displaying the analyzed responses.

In an embodiment of the invention, a user interface for real-time emotional state assessment is provided. Initially, the user is authenticated to access the emotional state assessment system. Upon authentication of a user, a cumulative annotated emotional state is displayed. The cumulative annotated information described herein include but are not limited to net happiness score, happiness index, net emotion score, net emotion index, attrition score, productivity score and safety score. The cumulative annotated information is presented as at least one of a graph, a pie-chart, a nested chart, a web-chart, radial chart, a spider chart, a bullet chart and using any relevant visual technique. Further, the user is presented with a plurality of filters to analyze the emotional state. The filters described herein include but are not limited to division, function, experience and location. The division allows selection of one or more departments within an organization, which includes but is not limited to Customer Service, Finance, Nursing, Research and Development, IT and Marketing. On selection of a division, all functions and locations associated with the selected division are displayed. The functions described herein include but are not limited to Delivery, Support, Accounts, Purchasing, Infrastructure and Operations, Enterprise Services, Corporate Law, Litigation, Market Research, Advertising, Dry Lab, Wet Lab, North, East, South, and West. The locations described herein include different branches of the organization. Upon selection of a filter, the user can assess the analyzed emotional state real-time. The assessment described herein includes but is not limited to a contextual assessment, comparative assessment, intervention assessment, productivity assessment, attrition assessment, performance assessment and project assessment. The cumulative annotated information presented is configured for display with respect to a pre-defined time interval, the pre-defined time interval is at least one of an hour, a day, a week or a month.

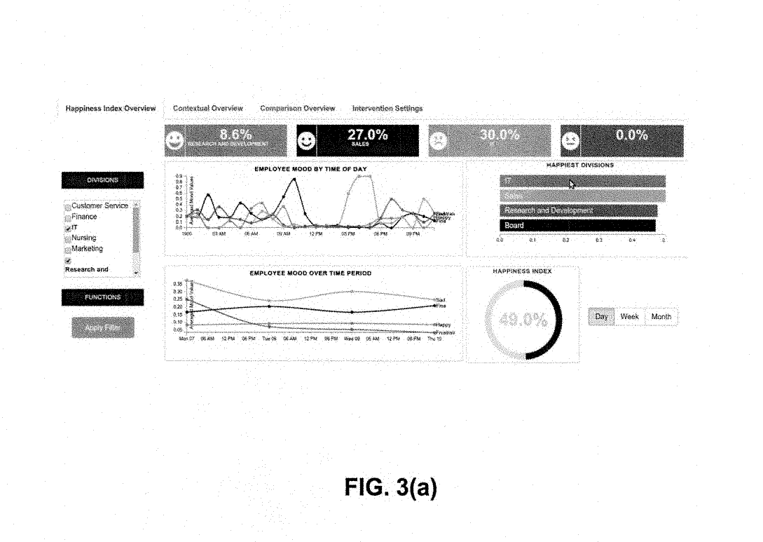

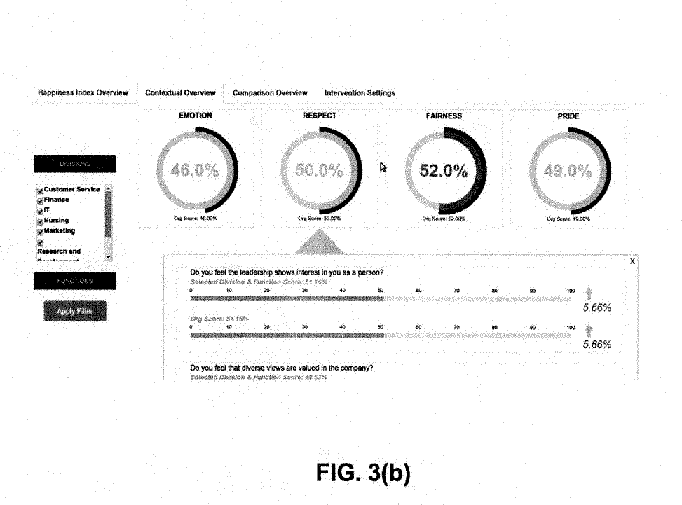

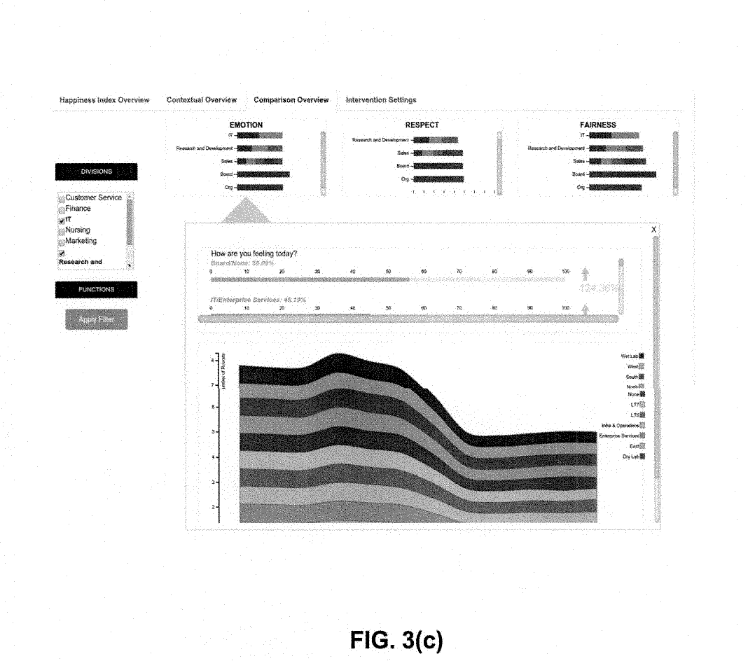

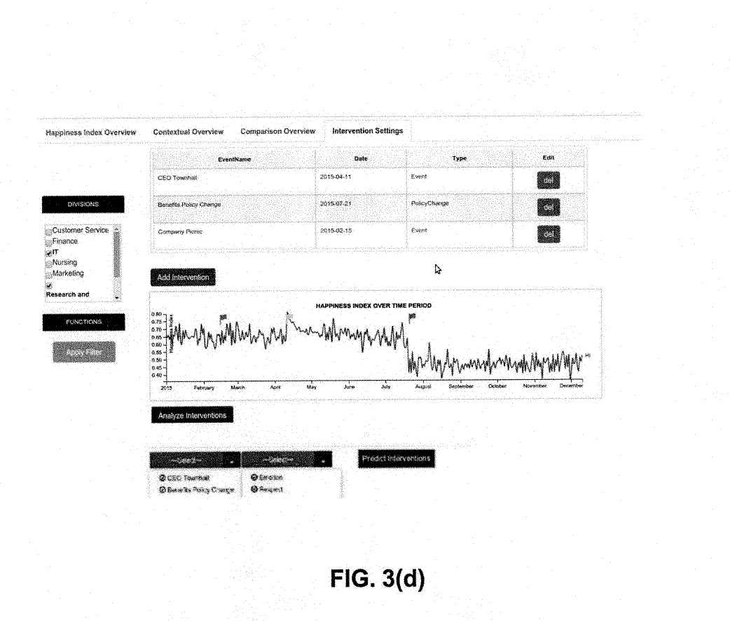

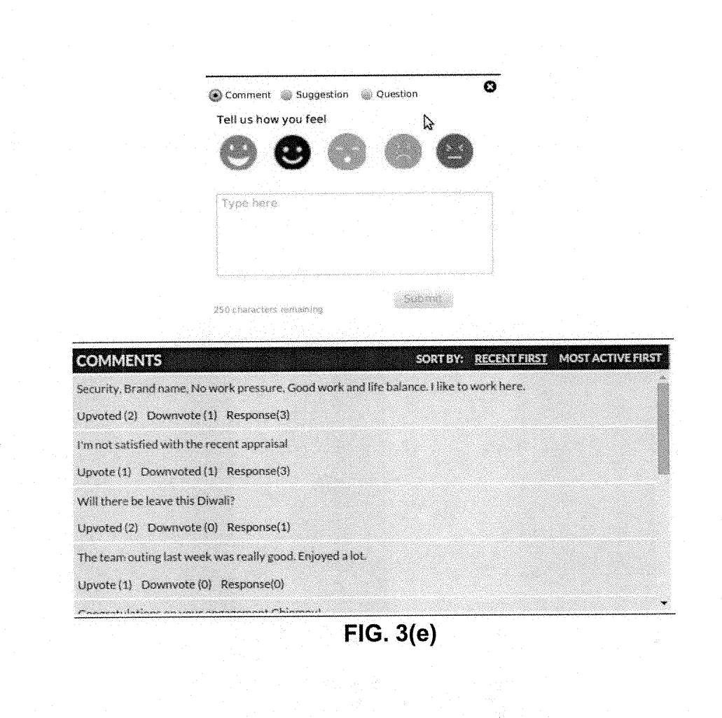

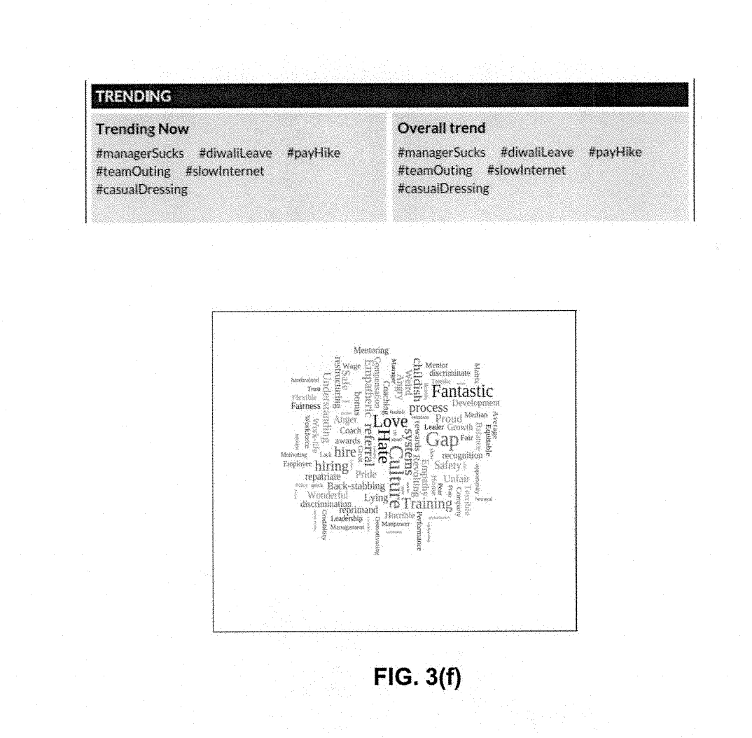

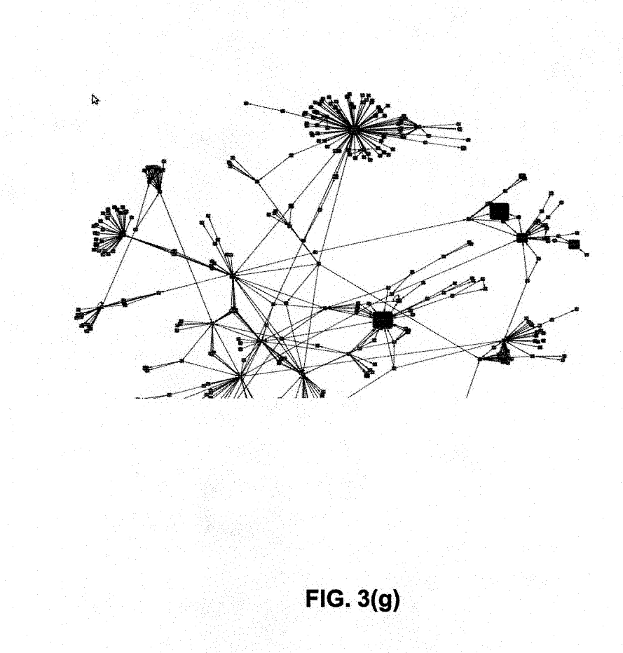

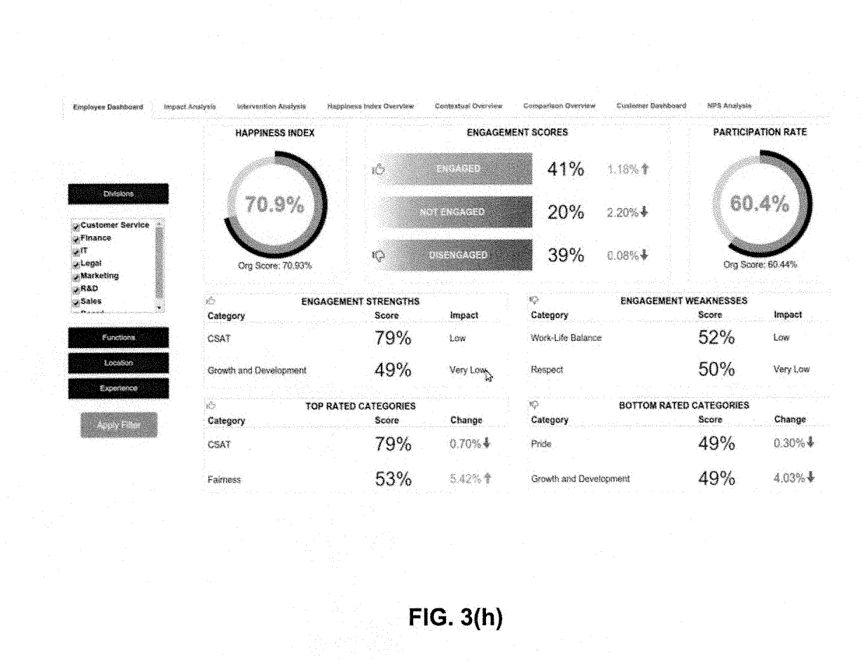

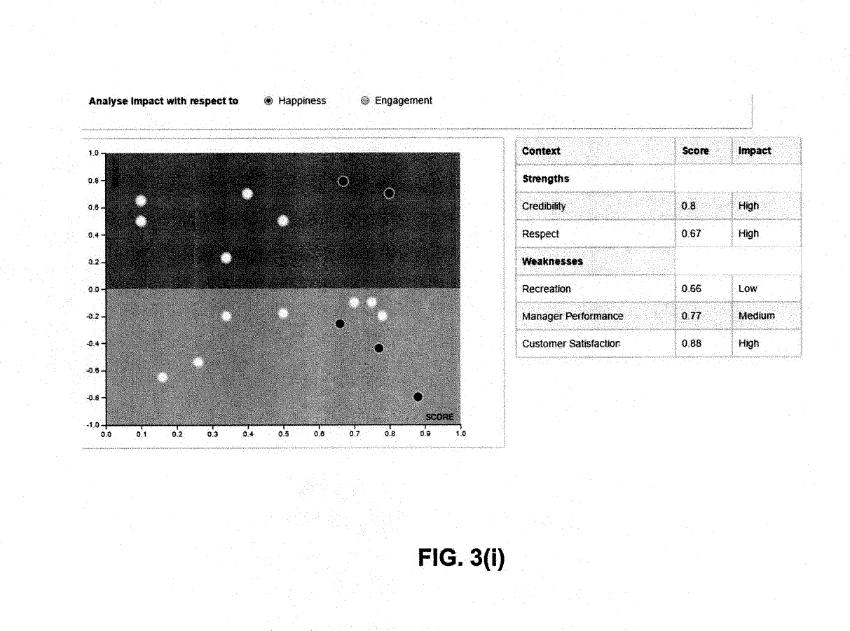

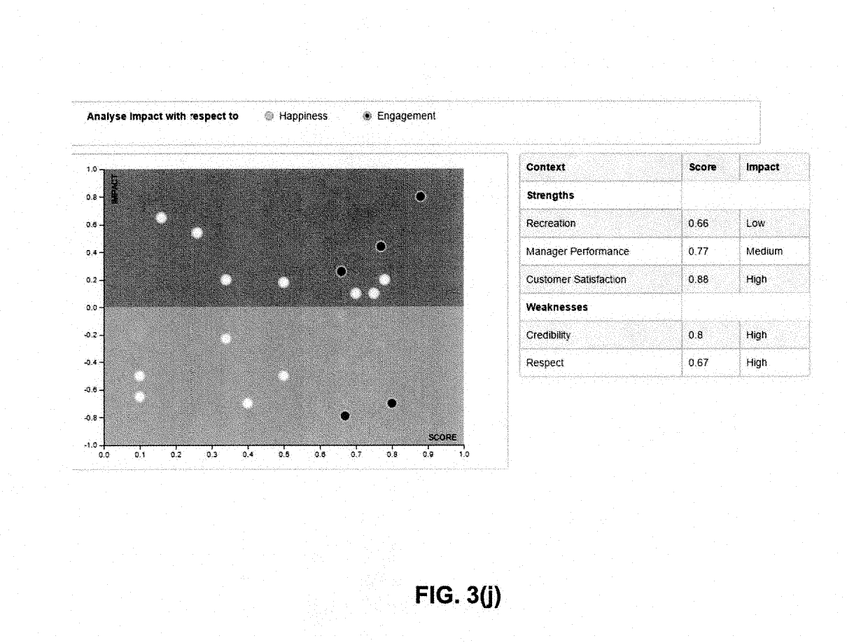

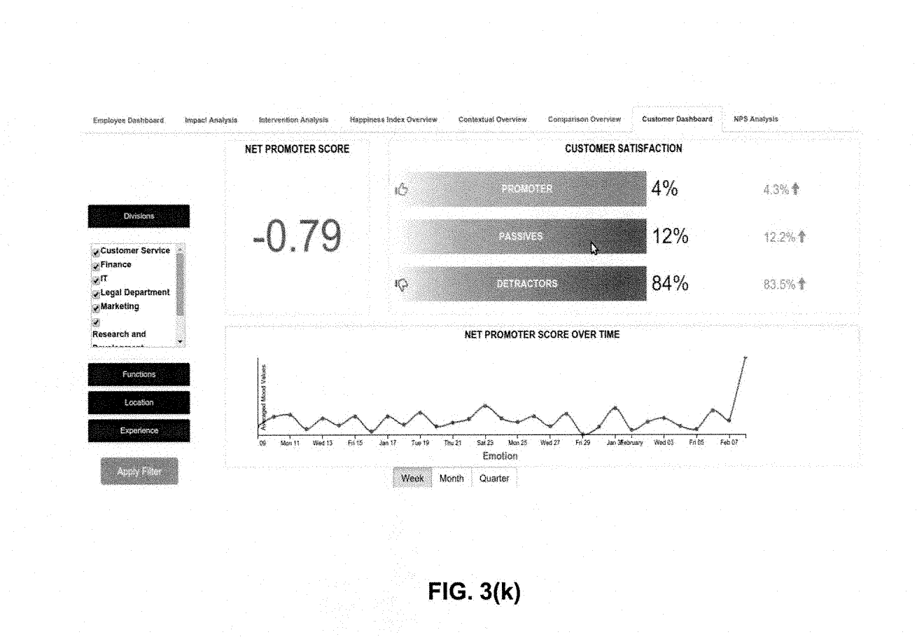

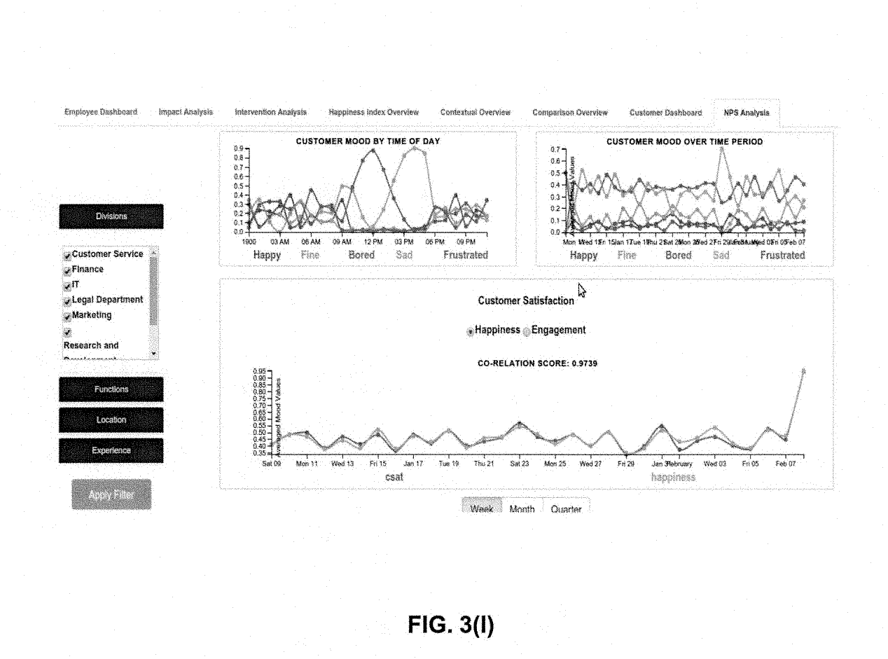

FIGS. 3(a)-3(l) generally describe graphical representation of user interface of the real-time emotional state assessment system. FIG. 3(a) illustrates a happiness index overview of the real-time emotional state assessment system, according to an embodiment of the invention. The happiness index overview provides views on leaders for each emotional state for a selected time period. The time period described herein includes but is not limited to a day, a month or a year. The emotional state described herein includes but is not limited to affection, anger, angst, anguish, annoyance, anxiety, apathy, arousal, awe, boredom, confidence, contempt, contentment, courage, curiosity, depression, desire, despair, disappointment, disgust, distrust, dread, ecstasy, embarrassment, envy, euphoria, excitement, fear, frustration, gratitude, grief, guilt, happiness, hatred, hope, horror, hostility, hurt, hysteria, indifference, interest, jealousy, joy, loathing, loneliness, love, lust, outrage, panic, passion, pity, pleasure, pride, rage, regret, remorse, sadness, satisfaction, schadenfreude, self-confidence, shame, shock, shyness, sorrow, suffering, surprise, trust, wonder, worry, zeal and zest.

Further, the happiness index overview shows overall happiness index for the selected filters, employee moods by time of day and employee moods over the selected time periods.

FIG. 3(b) illustrates a contextual overview of the real-time emotional state assessment system, according to an embodiment of the invention. The contexts include but are not limited to Fairness, Credibility, Trust, Growth and Development, Pay and Benefits, Work-Life Balance, Stress, Leadership, Manager Performance, Peer Group, Culture, Recreation, Diversity, Communication, Customer Orientation, Quality Emphasis, Training, Involvement and Customer Satisfaction. The radial charts or visual representation show emotional state value for the selected divisions and functions along with organizational value for comparison. The emotional state value can be represented as engagement score, performance score, productivity score, attrition score and any relevant metric that represents productivity, performance, training, leadership development and engagement. The selection of a radial chart opens up a sliding window that shows different questions that were asked to get response from the users for that particular contextual view. The responses for each question are shown along with the overall response at an organizational level for comparison. Month on month changes are also shown with green/red colored arrows along with the percentage changes.

FIG. 3(c) illustrates a comparison overview of the real-time emotional state assessment system, according to an embodiment of the invention. The comparison overview allows organizations to carry out a comparative analysis across divisions and functions for the different contexts chosen by the organization. The contexts described herein include but are not limited to Fairness, Credibility, Trust, Growth and Development, Pay and Benefits, Work-Life Balance, Stress, Leadership, Manager Performance, Peer Group, Culture, Recreation, Diversity, Communication, Customer Orientation, Quality Emphasis, Training, Involvement and Customer Satisfaction. The row charts at the top show the divisional level feedback for each emotional state. The selection of a radial chart opens up a sliding window that shows different questions that were asked to get feedback from the users for that particular context. The organizational feedback is given for comparative analysis along with month on month changes. Month on month changes are shown with green/red colored arrows along with the percentage changes. The chart at the bottom shows feedback levels for each context across all the functions for the course of a year. This gives organizations the ability to see relative differences across time.

FIG. 3(d) illustrates Intervention settings of the real-time emotion state assessment system, according to an embodiment of the invention. Intervention settings allow a person concerned with the user to add specific events. The person described herein is a person who performs assessment, which includes but is not limited to a Manager, a CXO, a Team leader, a consumer marketing professional, a customer service associate, a customer experience leader, a human resource associate and a business partner or a line manager. The events include but are not limited to weather forecast, sales promotion, pay-hike, fashion trend, political event, maternity leave, lay-off, CEO townhall, policy change and company picnic. The interventions can also be deleted if they are no longer required or have been wrongly entered by using the DEL button.

Clicking on the “Analyze Interventions” button, brings up flags on the chart with a green flag indicating a significant positive, red flag indicating significant negative and a grey flag indicating a neutral outcome from an intervention. The Intervention settings also provide an option to simulate interventions and predict the possible impact of these interventions on the various contexts of interest. The contexts of interest described herein include but are not limited to Fairness, Credibility, Trust, Growth and Development, Pay and Benefits, Work-Life Balance, Stress, Leadership, Manager Performance, Peer Group, Culture, Recreation, Diversity, Communication, Customer Orientation, Quality Emphasis, Training, Involvement and Customer Satisfaction. Clicking on the “Predict Interventions” button predicts what would happen in case such interventions are carried out based on the past history of the organization.

FIG. 3(e) illustrates a graphical representation of text based feedback, according to an embodiment of the invention. The users are provided with an ability to send written, anonymous feedback through the interface. The textual data is stored and visible to users through a web browser. Users can show their liking or disliking of the comments using the Upvote/Downvote buttons. Moreover, users can respond to the comments/suggestions/questions all in an anonymous manner. The messages are threaded for ease of usage.

FIG. 3(f) illustrates a graphical representation of trending, according to an embodiment of the invention. The textual feedback is analysed using a multitude of methods to show trending topics over a period of time. In an example of the invention, a sentiment analysis is performed to derive various representations of the data, including but not limited to a word cloud that delineates positive, very positive, negative and very negative.

FIG. 3(g) illustrates a text network, according to an embodiment of the invention. In an embodiment of the invention, groups of words that occur together are identified and networks of words are built around specific themes of interest. The themes of interest described herein include but are not limited to Fairness, Credibility, Trust, Growth and Development, Pay and Benefits, Work-Life Balance, Stress, Leadership, Manager Performance, Peer Group, Culture, Recreation, Diversity, Communication, Customer Orientation, Quality Emphasis, Training, Involvement and Customer Satisfaction. Each “node” in the network is a word and the links between words indicate the co-occurrence of these words.

FIG. 3(h) illustrates a representative screenshot of employee dashboard, according to an embodiment of the invention. The employee dashboard provides overall happiness index, engagement scores, strengths and weaknesses of engagement, participation rate, top rated categories and bottom rated categories. The categories described herein include but are not limited to Fairness, Credibility, Trust, Growth and Development, Pay and Benefits, Work-Life Balance, Stress, Leadership, Manager Performance, Peer Group, Culture, Recreation, Diversity, Communication, Customer Orientation, Quality Emphasis, Training, Involvement and Customer Satisfaction.

FIG. 3(i) illustrates strengths and weaknesses of employee happiness, according to an embodiment of the invention. The strengths and weaknesses of employee happiness are derived through impact analysis. In an example of the invention, impact analysis includes correlation analysis.

FIG. 3(j) illustrates strengths and weaknesses of employee engagement, according to an embodiment of the invention. The strengths and weaknesses of employee engagement are derived through impact analysis. In an example of the invention, impact analysis includes correlation analysis.

FIG. 3(k) illustrates a representative screenshot of a customer dashboard, according to an embodiment of the invention. The customer dashboard provides a net promoter score. The net promoter score is derived through user responses for specific customer related contexts. The customer related contexts described herein includes but are not limited to Credibility, Trust, Manager Performance, Culture, Recreation, Diversity, Communication, Customer Orientation, Quality Emphasis, Training, Involvement and Customer Satisfaction.

FIG. 3(l) illustrates a representative screenshot of NPS Analysis, according to an embodiment of the invention. In an example of the invention, the NPS Analysis provides the level of customer satisfaction through correlation analysis. The customer mood by time of the day, the customer mood over a time period and the averaged mood values are displayed.

Thus, the invention provides a method, system and user interface for real-time assessment of an emotional state. The applications of the invention includes Employee Engagement, Business Customer engagement, Consumer Engagement, Social Engagement, Public Engagement, Employee Survey, Business Customer Survey, Public Survey, Social Survey, Employee Voting, Public Voting, Business Customer Voting, Social Voting, Early warning system for Employee Behaviors, Early warning system for Manager Behaviors, Early warning system for Consumer Behaviors, Early warning system for business behaviors, Automated Employee Interventions, Automated Consumer Interventions and Automated Business Interventions. The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

We claim:

1. A method for real time assessment of an emotional state of users in a network, the method comprising:
Identifying an event in the network;
presenting a status specific to the event to the plurality of users;
receiving a response from each of the users corresponding to the presented status;
analyzing the responses received to obtain a cumulative annotated emotional state; and
displaying the annotated emotional state for assessment of the status presented.

2. The method of claim 1, wherein the status is presented to the plurality of users iteratively over pre-determined intervals of time.

3. The method of claim 1, wherein the network is a closed group network or an open network.

4. The method of claim 1, wherein the event is selected from a list comprising of a weather forecast, a sales promotion, a pay-hike, a fashion trend, a political event, a maternity leave, a lay-off, customer centricity, Manager Performance, Peer Group, Culture, Recreation, Diversity, Communication, Customer Orientation, Quality Emphasis, Training and Customer Satisfaction.

5. The method of claim 1, wherein the user is at least one selected from a list comprising of employees, contract workers, customers, social media consumers, social employee networks and general public.

6. The method of claim 1, wherein the response from the user is an emotional state which is at least one selected from a list comprising of happy, sad, anxiety, boredom, anger and excitement.

7. The method of claim 1, wherein the response is submitted as at least one of a picture, a text or an emoticon.

8. The method of claim 1, wherein the analysis of the response submitted is performed based on a plurality of parameters derived from the response.

9. The method of claim 1, wherein the parameter is at least one selected from a list comprising of nature of the user, time of response submission, date of response submission and location of user.

10. The method of claim 1, wherein the cumulative annotated emotional state is at least one selected from a list comprising of net happiness score, happiness index, net emotion score, net emotion index, net promoter score, attrition score, productivity score and safety score.

11. The method of claim 1, wherein the cumulative annotated information is presented as at least one selected from a list comprising of a graph, a table, a pie-chart, a nested chart, a web-chart, radial chart, spider chart and bullet chart.

12. The method of claim 1, wherein the assessment is performed by at least one person concerned with the user.

13. The method of claim 12, wherein the person is selected from a list comprising of Managers, CXOs, Team leaders, consumer marketing professional, customer service associate, customer experience leaders, human resource associate and business partners.

14. A system for real-time assessment of emotional state, the system comprising:
a first device configured for broadcasting a status;
at least one second device capable of receiving the broadcasted status wherein the second device is configured for submitting a response with respect to the broadcasted status;
an analyzer communicatively coupled to the first device and the second device wherein the analyzer is configured for analyzing the plurality of responses received; and
a display means coupled to the analyzer for displaying the analyzed responses.

15. The system of claim 14, wherein the first device is at least one of a server, a desktop, a laptop or a handheld device.

16. The system of claim 14, wherein the second device is a handheld device comprising of a smartphone, a tablet, a fablet or a wearable device.

17. The system of claim 14, wherein the broadcast of status by the first device is a push notification.

18. The system of claim 14, wherein the submitting of response by the second device is a push notification.

19. The system of claim 14, wherein the analyzer comprises of:
a database configured for retrievably storing the plurality of responses;
an evaluator engine communicatively coupled to the database; and
a presentation module;

20. The analyzer of claim 19, wherein the database is a physical storage or a virtual storage.

21. The analyzer of claim 19, wherein the evaluator engine is configured for real time analysis of the responses received.

22. The analyzer of claim 19, wherein the presentation module is configured to present the analyzed responses in at least one format selected from a list comprising of a table, a static graph, an index, an interactive graph, a pie-chart.

23. A user interface for real-time emotional state assessment, the user interface comprising:
means for authenticating the user;
displaying a cumulative annotated emotional state upon authentication of the user;
presenting the user with a plurality of filters to analyse the emotional state; and
assessing the analysed emotional state real-time;

24. The user interface of claim 23, wherein the filter is at least one selected from a list comprising of division, function, experience and location.

25. The user interface of claim 23, wherein the cumulative annotated information is at least one selected from a list comprising of net happiness score, happiness index, net emotion score, net emotion index, net promoter score, attrition score, productivity score and safety score.

26. The user interface of claim 23, wherein the cumulative annotated information is presented as at least one selected from a list comprising of a graph, a pie-chart, a nested chart, a web-chart, radial chart, spider chart and bullet chart.

27. The user interface of claim 23, further wherein the cumulative annotated information presented is configured for display with respect to a pre-defined time interval, the pre-defined time interval is at least one of an hour, a day, a week or a month.
